(19) United States
(12) Patent Application Publication
(10) Pub. No.: US 2024/0148764 A1
(43) Pub. Date: May 9, 2024

(54) INHIBITORS OF SGLT-1 AND USES THEREOF

(71) Applicants: The Regents of the University of Michigan, Ann Arbor, MI (US); Wayne State University, Detroit, MI (US)

(72) Inventors: Jie Xu, Ann Arbor, MI (US); Kezhong Zhang, DETROIT, MI (US); Xiubin Liang, Ann Arbor, MI (US); Yuqing Eugene Chen, Ann Arbor, MI (US)

(21) Appl. No.: 18/280,293

(22) PCT Filed: Mar. 4, 2022
(86) PCT No.: PCT/US2022/018958

§ 371 Date: Sep. 5, 2023

Related U.S. Application Data
(60) Provisional application No. 63/157,435, filed on Mar. 5, 2021.

(51) Int. Cl. A61K 31/7034; A61K 31/7048; A61K 31/7056; A61P 1/16

(52) U.S. Cl. CPC A61K 31/7034; A61K 31/7048; A61K 31/7056; A61P 1/16

(57) ABSTRACT

This invention is in the field of medicinal pharmacology. In particular, the present invention relates to pharmaceutical agents which function as inhibitors of sodium-glucose cotransporter 1 (SGLT-1) activity. The invention further relates to methods of treating and/or ameliorating symptoms related to cystic fibrosis-related liver disease and diseases characterized with increased SGLT-1 activity, increased endoplasmic reticulum (ER) stress response, and/or increased hepatic inflammation.

### CROSS-REFERENCE TO RELATED APPLICATION

This application claims the priority benefit of U.S. Provisional Application No. 63/157,435, filed Mar. 5, 2021 which is incorporated herein by reference in its entirety.

### STATEMENT REGARDING FEDERALLY SPONSORED RESEARCH OR DEVELOPMENT

This invention was made with government support under HL133162 awarded by the National Institutes of Health. The government has certain rights in the invention.

### FIELD OF THE INVENTION

This invention is in the field of medicinal pharmacology. In particular, the present invention relates to pharmaceutical agents which function as inhibitors of sodium-glucose cotransporter 1 (SGLT-1) activity. The invention further relates to methods of treating and/or ameliorating symptoms related to cystic fibrosis-related liver disease and diseases characterized with increased SGLT-1 activity, increased endoplasmic reticulum (ER) stress response, and/or increased hepatic inflammation.

### INTRODUCTION

Cystic fibrosis (CF) is an autosomal genetic disease caused by mutations in the cystic fibrosis transmembrane conductance regulator (CFTR) gene (1). CF-related liver disease (CFLD) is the third-leading cause of mortality in CF (2). The disease is complex because it affects multiple organs involving the epithelia of the respiratory tract, exocrine pancreas, intestine, hepatobiliary system, and sweat glands, in which CFTR is expressed and has a critical function. There has been major progress in understanding CF pathogenesis since the cloning of the CFTR gene in 1989. In October 2019, FDA approved Trikafta, a combination of CFTR potentiator VX-770 and CFTR correctors VX-445 and VX-661, which provides benefits to >90% of CF patients (3). However, many important questions remain unanswered related to disease pathogenesis in numerous organs and organ-level responsiveness to therapy.

CF-associated liver disease (CFLD) is a major nonpulmonary cause of mortality in CF, with about one third CF patients suffer from it (2). Clinical manifestations of CFLD are heterogeneous, including cholestasis, focal biliary cirrhosis, hepatic steatosis, fibrosis, and the presence of a microgallbladder. The peak of CFLD is in the pediatric population, but a second wave of liver disease in CF adults has been reported in the past decade in association with an increase in the life expectancy of these patients. In the post-Trikafta era, nonpulmonary CF diseases such as CFLD rise in the priority list demanding novel and effective therapeutics.

To date, ursodeoxycholic acid (UDCA) is the only medicine that has gained FDA approval for treating CFLD. However, at least according to some clinicians and researchers, the efficacy of UDCA on CFLD remain controversial, because there is a lack of convincing data from randomized controlled trials assessing hard endpoints such as improvement in liver histology, mortality or liver transplant free survival (4). Trikafta, despite its remarkable benefits on the lung functions, increase the levels of key liver function enzymes ALT, AST and others. In fact, adverse effects on liver functions are major side effect concerns of this drug, raising concerns of aggravating CFLD.

Accordingly, improved methods and techniques are needed for treating and/or ameliorating CFLD and related clincial manifestations.

The present invention addresses this need.

### SUMMARY

Sodium-glucose cotransporter (SGLT) inhibitors, including selective SGLT2 inhibitors and dual SGLT1/2 inhibitors, have becoming mainstream therapy for diabetes. As of today, the effects of SGLT inhibitors in liver disease have not been systematically tested. In experiments conducted during the course of developing embodiments for the present invention, a rabbit model of cystic fibrosis liver disease (CFLD) was utilized to examine the effects of SGLT inhibitor drugs on liver diseases. CFLD-like phenotypes in the CF rabbits include spontaneous hepatobiliary lesions, increased liver damage and Non-Alcoholic SteatoHepatitis (NASH) activities, and altered lipid and glucose homeostasis. Experiments identified age associated activation of endoplasmic reticulum (ER) stress response or unfolded protein response (UPR) mediated through the UPR transducer IRE1α and its downstream transcription factor spliced XBP1 (XBP1s), and activation of NF-kB inflammatory pathway in CF rabbit livers. SGLT1 and SGLT2 expression levels in CF and WT rabbits were determined, and it was revealed that the SGLT2 expression, like that reported in humans, is largely restricted to the kidney, and there is no difference in between CF and WT rabbits; whereas SGLT1 expression was shown to be elevated in CF rabbit liver and other organs. Experiments were next conducted that proceeded to treat CF rabbits with a SGLT1/2 dual inhibitor drug Sotagliflozin (Sota, 15 mg/kg/day) by daily gavage for 4 weeks. Sota treatment exerted surprisingly beneficial effects to CF rabbits by increasing body weight and life span, restoring blood glucose homeostasis, and improving liver functions. Importantly, Sota treatment mitigated hepatic ER stress and inflammatory responses and attenuated hepatic and metabolic dysregulation in CF rabbits. Such results indicate that an SGLT inhibitor drug such as Sota has beneficial effects on liver disease exampled here such as CFLD, through suppression of glucose transport attenuates hepatic inflammatory stress response, hence improve NASH state, and ameliorates liver disease phenotypes.

Accordingly, the present invention relates to pharmaceutical agents which function as inhibitors of sodium-glucose cotransporter 1 (SGLT-1) activity. The invention further relates to methods of treating and/or ameliorating symptoms related to cystic fibrosis-related liver disease and diseases characterized with increased SGLT-1 activity, increased endoplasmic reticulum (ER) stress response, and/or increased hepatic inflammation.

In certain embodiments, the present invention provides compositions comprising a pharmaceutical agent capable of inhibiting SGLT-1 activity.

In certain embodiments, the present invention provides methods for inhibiting the activity of SGLT-1 in a subject, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity, increased endoplasmic reticulum (ER) stress response, and/or increased hepatic inflammation (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and hepatocellular carcinoma (HCC)).

In certain embodiments, the present invention provides methods for treating, ameliorating and/or preventing in a subject one or more liver diseases and/or conditions characterized by increased SGLT-1 activity, increased endoplasmic reticulum (ER) stress response, and/or increased hepatic inflammation, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC).

In certain embodiments, the present invention provides methods for treating, ameliorating and/or preventing in a subject one or more symptoms related to liver diseases and/or conditions associated with increased SGLT-1 activity, increased endoplasmic reticulum (ER) stress response, and/or increased hepatic inflammation, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC).

Such methods are not limited to treating, ameliorating and/or preventing specific symptoms related to liver diseases and/or conditions associated increased SGLT-1 activity, increased ER stress, and/or increased hepatic inflammation. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing CFLD in a subject, comprising administering to the subject a composition comprising a pharmaceutical agent capable of inhibiting SGLT-1 activity. In some embodiments, the subject is a human subject. In some embodiments, the subject is a human subject suffering from CFLD. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing spontaneous hepatobiliary lesions in a subject, comprising administering to the subject a composition comprising a pharmaceutical agent capable of inhibiting SGLT-1 activity. In some embodiments, the subject is a human subject. In some embodiments, the subject is a human subject suffering from spontaneous hepatobiliary lesions. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing liver damage in a subject, comprising administering to the subject a composition comprising a pharmaceutical agent capable of inhibiting SGLT-1 activity. In some embodiments, the subject is a human subject. In some embodiments, the subject is a human subject suffering from liver damage. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing NASH in a subject, comprising administering to the subject a composition comprising a pharmaceutical agent capable of inhibiting SGLT-1 activity. In some embodiments, the subject is a human subject. In some embodiments, the subject is a human subject suffering from NASH. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing altered lipid and glucose homeostasis in a subject, comprising administering to the subject a composition comprising a pharmaceutical agent capable of inhibiting SGLT-1 activity. In some embodiments, the subject is a human subject. In some embodiments, the subject is a human subject suffering from altered lipid and glucose homeostasis. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing hepatic ER stress and related inflammatory responses in a subject, comprising administering to the subject a composition comprising a pharmaceutical agent capable of inhibiting SGLT-1 activity. In some embodiments, the subject is a human subject. In some embodiments, the subject is a human subject suffering from hepatic ER stress and related inflammatory responses. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing hepatic and metabolic dysregulation in a subject, comprising administering to the subject a composition comprising a pharmaceutical agent capable of inhibiting SGLT-1 activity. In some embodiments, the subject is a human subject. In some embodiments, the subject is a human subject suffering from hepatic and metabolic dysregulation. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides kits comprising (1) a composition comprising a pharmaceutical agent capable of inhibiting SGLT-1 activity, (2) a container, pack, or dispenser, and (3) instructions for administration.

Such compositions, methods, and kits are not limited to a particular type or kind of pharmaceutical agent capable of inhibiting SGLT-1 activity. In some embodiments, the pharmaceutical agent capable of inhibiting SGLT-1 activity is a small molecule, an antibody, nucleic acid molecule (e.g., siRNA, antisense oligonucleotide, an aptamer), or a mimetic peptide. In some embodiments, the pharmaceutical agent capable of inhibiting SGLT-1 activity is selected from, but not limited to, for example, Phlorizin, Canagliflozin ((2S,3R,4R,5S,6R)-2-{3-[5-[4-Fluoro-phenyl)-thiophen-2-ylmethyl]-4-methyl- -phenyl}-6-hydroxymethyl-tetrahydro-pyran-3,4,5-triol), Dapagliflozin ((2S,3R,4R,5S,6R)-2-[4-chloro-3-(4-ethoxybenzyl)phenyl]-6-(hydroxymethyl)-tetrahydro-2H-pyran-3,4,5-triol), Empagliflozin ((2S,3R,4R,5S,6R)-2-[4-chloro-3-[[4-[(3S)-oxolan-3-yl]oxyphenyl]methyl]ph-enyl]-6-(hydroxymethyl)oxane-3,4,5-triol), Remogliflozin (5-methyl-4-[4-(1-methylethoxy)benzyl]-1-(1-methylethyl)-1H-pyrazol-3-yl 6-O-(ethoxycarbonyl)-(3-D-glucopyranoside), Sergliflozin (2-(4-methoxybenzyl)phenyl 6-O-(ethoxycarbonyl)-(3-D-glucopyranoside), and Tofogliflozin ((1S,3′R,4′S,5′S,6′R)-6-(4-Ethylbenzyl)-6′-(hydroxymethyl)-3′,4′,5′,6′-te-trahydro-3H-spiro[2-benzofuran-1,2′-pyran]-3′,4′,5′-triol hydrate (1:1)), and Sotagliflozin (2S,3R,4R,5S,6R)-2-(4-chloro-3-(4-ethoxybenzyl)phenyl)-6-(methylthio)tetrahydro-2H-pyran-3,4,5-triol (LX4211), or a pharmaceutically acceptable salt thereof.

### BRIEF DESCRIPTION OF THE DRAWINGS

FIG. 1: Selected typical CF phenotypes in CF rabbits. (A) % survival of CF rabbits with (orange dots) or without GI laxative (blue), in comparison to WT (black). (B) Typical defective short circuit response of CF rabbit intestinal epithelial cells (grey bars) vs the normal response of WT ones (white bars). Note the elevated grey bars (CF) after glucose and phloridzin (a SGLT1/2 absence of feces pellets) but not observed in WT (left) rabbits. Arrowhead: point of blockage.

FIG. 2: CF rabbits display hepatobiliary lesions and abnormal biliary secretion. HE (A) and Sirius-red collagen (B) staining of liver sections of CF and WT rabbits of ˜60 days of age showing the typical biliary cirrhosis (arrows) and mucus plug in CF rabbits. (C) Microscopy images of bile from gallbladders of CF and WT rabbits on cover-slides. Mucus and pigment stones (arrows) appeared in bile of CF rabbits. (D-F) Bile pH values, levels of total serum bile acids, and relative serum bile protein abundance (fold changes CF vs WT). Mean±SEM (n=5 WT or 10 CF). *p≤0.05; **p≤0.01.

FIG. 3: CF rabbits display micro-gallbladders and liver injuries. (A) Micro-bladders in a CF rabbit vs a WT rabbit of ˜60 days old. (B) HE staining of liver sections showing cirrhosis in biliary triad (arrows) in CF rabbits. (C) Serum levels of ALT & AST in WT and CF rabbits. (D) Relative liver weights of WT and CF rabbits (% liver weight/body weight). n=7 for C; n=4 for D. *p≤0.05; **p≤0.01.

FIG. 4: CF rabbits display NASH phenotype. (A) Histological analysis of liver cellular structure (HE staining), lipid accumulation (oil-red O staining), and collagen fiber (Gomori's trichrome staining) in liver tissue sections from WT and CF rabbits. Arrows point to areas of hepatic inflammation or fibrosis. (B) Scoring for NASH activities in CF and WT rabbit livers based on the modified Brunt scoring system (17).

FIG. 5: CF rabbits show elevated lipid profiles and up-regulation of hepatic metabolic regulators. (A) Levels of plasma cholesterol (TC), triglycerides (TG), high-density lipoprotein (HDL) and low-density lipoprotein (LDL) in WT and CF rabbits of ˜60 days of age. Mean±SEM (n=9). (B) Ratios of LDL vs HDL. (C) Western blot analyses and quantification of CREBH, PPARα and FGF21 protein levels in WT and CF of different age (days after born). *p≤0.05; **p≤0.01.

FIG. 6: CF rabbits show glucose intolerance and diminished glycogen storage. (A-C) Levels of blood glucose, insulin and body weights of WT and CF rabbits of 60 days of age. (D-E) IVGTT analyses of CF, CFLD-like, and WT control rabbits. (F-G) PAS staining and enzymatic assay of hepatic glycogens in WT and CF rabbits (n=7). *p≤0.05; **p≤0.01.

FIG. 7: Inflammatory responses through JNK and NFκB are activated in CF rabbit livers. Western blot analyses and quantification of phosphorylated JNK (P-JNK), total JNK, phosphorylated IκB (P-IκB), and total IκB protein in WT and CF of different age (days after born). The graph shows fold changes in protein levels based on the intensity of protein signals, determined by Western blot densitometry. Mean±SD (n=6 WT & 9 CF). *p≤0.05; **p≤0.01.

FIG. 8: ER stress signaling is activated in CF rabbit livers. (A) Western blot analyses of IRE1α protein levels in WT and CF of different age. (B) IHC staining of IRE1α and XBP1 with liver sections from WT and CF rabbits of ˜60 days. (C-D) qPCR analyses of the mRNAs encoding ER stress sensor or mediators in ER stress response or ERAD pathways in the livers of WT and CF rabbits (n=4). *p≤0.05; **p≤0.01.

FIG. 9: SGLT1 in CF rabbits. Int: intestine. Pan: pancreas.

FIG. 10: SGLT1 in human CF cells. (A) SGLT1 and CFTR in CFBE-WT and CFBE-dF cells. VX809 and low temperature (27° C.) were used to rescue CFTR-dF. (B) SGLT1 and CFTR in iPSC derived lung organoids of WT, dF/dF or dF/G551D genotypes. (C) Single cell RNA sequencing from secretory airway cells from CF patients.

FIG. 11: IVGTT analysis of CF rabbits before and after Sota treatment. *p≤0.05.

FIG. 12: Selected blood chemistry of CF rabbits treated (green dots) or not treated (red dots) with Sota. Grey box: normal range. X-axis shows weeks post Sota treatment.

FIG. 13: Sota promotes appetite (left) and weight gain (right) of CF rabbits.

FIG. 14: Sota elongates lifespan of CF rabbits.

FIG. 15: Sota decreased SGLT1 and major ER stress and inflammatory mediators in CF rabbit livers. Levers of SGLT1 (A), HRD1, XBP1s, and phosphorylated NF-κB P65 (B) proteins in the livers of CF or WT rabbits after Sota treatment (15 mg/kg/day) for 4 weeks determined by Western blot analyses. (C-D) qPCR analyses of mRNA levels of Xbp1s, GRP78, TNFα and IL6. *p≤0.05.

FIG. 16: SGLT1 is expressed in Albumin positive cells in CF rabbit liver.

FIG. 17: HE, Sirius-red and PAS staining of rabbit livers.

FIG. 18: BA species in the liver (B) samples from WT and CF rabbits treated with or w/o Sota. *P≤0.05. **P≤0.01.

FIG. 19: Sota decreased SGLT1 and major ER stress and inflammatory mediators in CF rabbit livers. (A) Protein levels of SGLT1, HRD1, XBP1s, and phosphorylated NF-κB P65 (B) Transcription levels of SLC5A1 and IRE1α in the livers of CF or WT rabbits after Sota. (C) HE and IHC staining of the liver of CF rabbits treated with/without Sota.

FIG. 20: XBP1s is a transcription factor of SLC5A1. (A) Overexpression of XBP1s by Adv led to increased protein levels of SGLT1. (B) Overexpression of XBP1s increased SLC5A1 transcription. (C) ChiP assay confirm that XBP1s binds to the promoter of SLC5A1. (D) The putative binding motif (blue colored) on the promoter of SLC5A1. (E) SGLT1-luciferase assay show that mutant motif sequence abolishes the transcription activity induced by)(BP's.

FIG. 21: Sota (5 μg/ml) attenuated PA (10 μg/mL)-induced steatosis in Huh7 cells. Left: vehicle. PA: palmitate.

FIG. 22: Hypothesized mechanism of action of how SGLT1 inhibition benefits inflammatory liver diseases.

### DETAILED DESCRIPTION OF THE INVENTION

As such, this invention relates to pharmaceutical agents which function as inhibitors of sodium-glucose cotransporter 1 (SGLT-1) activity. The invention further relates to methods of treating and/or ameliorating symptoms related to cystic fibrosis-related liver disease and diseases characterized with increased SGLT-1 activity, increased endoplasmic reticulum (ER) stress response, and/or increased hepatic inflammation.

In certain embodiments, the present invention provides methods for inhibiting the activity of SGLT-1 in a subject, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC).

In certain embodiments, the present invention provides methods for treating, ameliorating and/or preventing in a subject one or more liver diseases and/or conditions characterized by increased SGLT-1 activity, increased endoplasmic reticulum (ER) stress response, and/or increased hepatic inflammation, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC).

In certain embodiments, the present invention provides methods for inhibiting the activity of SGLT-1 in a subject, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC).

In certain embodiments, the present invention provides methods for treating, ameliorating and/or preventing one or more liver diseases and/or conditions characterized by increased SGLT-1 activity in a subject, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC).

In certain embodiments, the present invention provides methods for treating, ameliorating and/or preventing one or more symptoms related to liver diseases and/or conditions associated with increased SGLT-1 activity in a subject, comprising administering to the subject a composition comprising a pharmaceutical agent capable of inhibiting SGLT-1 activity. In some embodiments, the subject is a human subject. In some embodiments, the subject is a human subject suffering from or at risk of suffering from one or more liver diseases and/or conditions characterized by increased SGLT-1 activity (e.g., CFLD, spontaneous hepatobiliary lesions, increased liver damage, NASH activities, altered lipid and glucose homeostasis, alpha-1 antitrypsin deficiency (AATD), chronic viral hepatitis (e.g., chronic Hepatitis C and Hepatitis B), cholestatic liver diseases (e.g., primary biliary cholangitis (PBC), primary sclerosing cholangitis (PSC), genetic liver diseases and biliary obstructions), alcohol-induced liver injury or alcoholic fatty liver disease, hyperhomocysteinemia, liver ischemia/reperfusion injury (e.g., liver ischemia/reperfusion (I/R) injury can occur during systemic hypotension, vascular occlusion, and surgery including liver transplantation), drug-induced liver injuries, and HCC). Such methods are not limited to treating, ameliorating and/or preventing specific symptoms related to liver diseases and/or conditions associated with increased SGLT-1 activity. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing CFLD in a subject, comprising administering to the subject a composition comprising a pharmaceutical agent capable of inhibiting SGLT-1 activity. In some embodiments, the subject is a human subject. In some embodiments, the subject is a human subject suffering from CFLD. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing spontaneous hepatobiliary lesions in a subject, comprising administering to the subject a composition comprising a pharmaceutical agent capable of inhibiting SGLT-1 activity. In some embodiments, the subject is a human subject. In some embodiments, the subject is a human subject suffering from spontaneous hepatobiliary lesions. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing liver damage in a subject, comprising administering to the subject a composition comprising a pharmaceutical agent capable of inhibiting SGLT-1 activity. In some embodiments, the subject is a human subject. In some embodiments, the subject is a human subject suffering from liver damage. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing NASH in a subject, comprising administering to the subject a composition comprising a pharmaceutical agent capable of inhibiting SGLT-1 activity. In some embodiments, the subject is a human subject. In some embodiments, the subject is a human subject suffering from NASH. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing altered lipid and glucose homeostasis in a subject, comprising administering to the subject a composition comprising a pharmaceutical agent capable of inhibiting SGLT-1 activity. In some embodiments, the subject is a human subject. In some embodiments, the subject is a human subject suffering from altered lipid and glucose homeostasis. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing hepatic ER stress and related inflammatory responses in a subject, comprising administering to the subject a composition comprising a pharmaceutical agent capable of inhibiting SGLT-1 activity. In some embodiments, the subject is a human subject. In some embodiments, the subject is a human subject suffering from hepatic ER stress and related inflammatory responses. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

In certain embodiments, the present invention provides methods for treating, ameliorating and/or preventing hepatic and metabolic dysregulation in a subject, comprising administering to the subject a composition comprising a pharmaceutical agent capable of inhibiting SGLT-1 activity. In some embodiments, the subject is a human subject. In some embodiments, the subject is a human subject suffering from hepatic and metabolic dysregulation. In some embodiments, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

The present invention is not limited to particular types or kinds of pharmaceutical agents which function as inhibitors of SGLT-1 activity. In some embodiments, the pharmaceutical agent capable of inhibiting SGLT-1 activity is a small molecule, an antibody, nucleic acid molecule (e.g., siRNA, antisense oligonucleotide, an aptamer), or a mimetic peptide.

In some embodiments, the pharmaceutical agent capable of inhibiting SGLT-1 activity is selected from but not limited to, for example, Phlorizin, Canagliflozin ((2S,3R,4R,5S,6R)-2-{3-[5-[4-Fluoro-phenyl)-thiophen-2-ylmethyl]-4-methyl- -phenyl}-6-hydroxy methyl-tetrahydro-pyran-3,4,5-triol), Dapagliflozin ((2S,3R,4R,5S,6R)-2-[4-chloro-3-(4-ethoxybenzyl)phenyl]-6-(hydroxymethyl)-tetrahydro-2H-pyran-3,4,5-triol), Empagliflozin ((2S,3R,4R,5S,6R)-2-[4-chloro-3-[[4-[(3S)-oxolan-3-yl]oxyphenyl]methyl]ph-enyl]-6-(hydroxymethyl)oxane-3,4,5-triol), Remogliflozin (5-methyl-4-[4-(1-methylethoxy)benzyl]-1-(1-methylethyl)-1H-pyrazol-3-yl 6-O-(ethoxycarbonyl)-(3-D-glucopyranoside), Sergliflozin (2-(4-methoxybenzyl)phenyl 6-O-(ethoxycarbonyl)-(3-D-glucopyranoside), and Tofogliflozin ((1S,3′R,4′S,5′S,6′R)-6-(4-Ethylbenzyl)-6′-(hydroxymethyl)-3′,4′,5′,6′-te-trahydro-3H-spiro[2-benzofuran-1,2′-pyran]-3′,4′,5′-triol hydrate (1:1)), and Sotagliflozin (LX4211), or a pharmaceutically acceptable salt thereof. An important aspect of the present invention is that the compositions of the present invention (e.g., compositions comprising pharmaceutical agents which function as inhibitors of SGLT-1 activity) are useful in treating one or more liver diseases and/or conditions characterized by increased SGLT-1 activity (e.g., CFLD) (e.g., CFLD-like phenotypes (e.g., spontaneous hepatobiliary lesions, increased liver damage, NASH activities, and altered lipid and glucose homeostasis).

Some embodiments of the present invention provide methods for administering an effective amount of a composition comprising a pharmaceutical agent which functions as an inhibitor of SGLT-1 activity of the invention and at least one additional therapeutic agent (including, but not limited to, any pharmaceutical agent useful in treating one or more liver diseases and/or conditions characterized by increased SGLT-1 activity (e.g., CFLD) (e.g., CFLD-like phenotypes (e.g., spontaneous hepatobiliary lesions, increased liver damage, NASH activities, and altered lipid and glucose homeostasis).

Compositions within the scope of this invention include all compositions wherein the pharmaceutical agents which function as inhibitors of SGLT-1 activity are contained in an amount that is effective to achieve its intended purpose. While individual needs vary, determination of optimal ranges of effective amounts of each component is within the skill of the art. Typically, the pharmaceutical agents which function as inhibitors of SGLT-1 activity (e.g., small molecules, antibodies, mimetic peptides) may be administered to mammals, e.g. humans, orally at a dose of 0.0025 to 50 mg/kg, or an equivalent amount of the pharmaceutically acceptable salt thereof, per day of the body weight of the mammal being treated for disorders responsive to inhibition of SGLT-1 activity. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 3000 mg, for example, about 0.1 to about 100 mg of the SGLT-1 activity inhibiting agent. The unit dose may be administered one or more times daily as one or more tablets or capsules or liquid or vaporized/inhalation form each containing from about 0.1 to about 10 mg, conveniently about 0.25 to 50 mg of the SGLT-1 activity inhibiting agent (e.g., mimetic peptide, small molecule) or its solvates.

In a formulation (e.g., intravenous formulation, intraperitoneal formulation, intramuscular formulation, subcutaneous formulation, injection formulation, topical formulation, oral formulation, etc.), the SGLT-1 activity inhibiting agent (e.g., mimetic peptide, small molecule) may be present at a concentration of about 0.01 to 100 mg per gram of carrier. In a one embodiment, the SGLT-1 activity inhibiting agent (e.g., mimetic peptide, small molecule) is present at a concentration of about 0.07-1.0 mg/ml, for example, about 0.1-0.5 mg/ml, and in one embodiment, about 0.4 mg/ml.

In addition to administering the SGLT-1 activity inhibiting agent (e.g., mimetic peptide, small molecule) as a raw chemical, SGLT-1 activity inhibiting agents (e.g., mimetic peptides, small molecule) of the invention may be administered as part of a pharmaceutical preparation containing suitable pharmaceutically acceptable carriers comprising excipients and auxiliaries which facilitate processing of the SGLT-1 activity inhibiting agents into preparations which can be used pharmaceutically. The preparations, particularly those preparations which can be administered in any desired manner (e.g., intravenous, intraperitoneal, intramuscular, subcutaneous, topical, oral, inhaled, etc.) and which can be used for one type of administration, such as tablets, dragees, slow release lozenges and capsules, inhalants, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of a SGLT-1 activity inhibiting agent (e.g., mimetic peptides, small molecules) of the invention. Foremost among such patients are mammals, e.g., humans, although the invention is not intended to be so limited. Other patients include veterinary animals (rabbits, cows, sheep, pigs, horses, dogs, cats and the like).

The SGLT-1 activity inhibiting agents (e.g., mimetic peptides, small molecules) and pharmaceutical compositions thereof may be administered by any means that achieve their intended purpose. For example, administration may be by parenteral, subcutaneous, intravenous, intramuscular, intraperitoneal, inhalation, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EXPERIMENTAL

The following examples are provided in order to demonstrate and further illustrate certain preferred embodiments and aspects of the present invention and are not to be construed as limiting the scope thereof. As used through the following experimental section, the term “we” or “our” or “us” or similar terms refers to one or more of the inventors.

### Example I

The following experiments present two lines of data: (1) development of CF rabbits and their liver phenotypes; and (2) beneficial effects of Sota on CF rabbits. CF rabbit models and their liver phenotypes.

### Development of CF Rabbit Models.

In the past several years, we (the inventors) have produced several lines of CFTR mutant rabbits by using CRISPR/Cas9 (13, 14). Relevant to the present work, the CFTRA9 mutation is a nine bp deletion that leads to three amino acids (P477, 5478 and E479) deletion in nucleotide binding domain 1 (NBD1), referred to as APSE in this proposal. The CFTR-ΔF508 rabbits (14) carry the most prevalent mutation found in human CF patients.

### General Features of CF Rabbits.

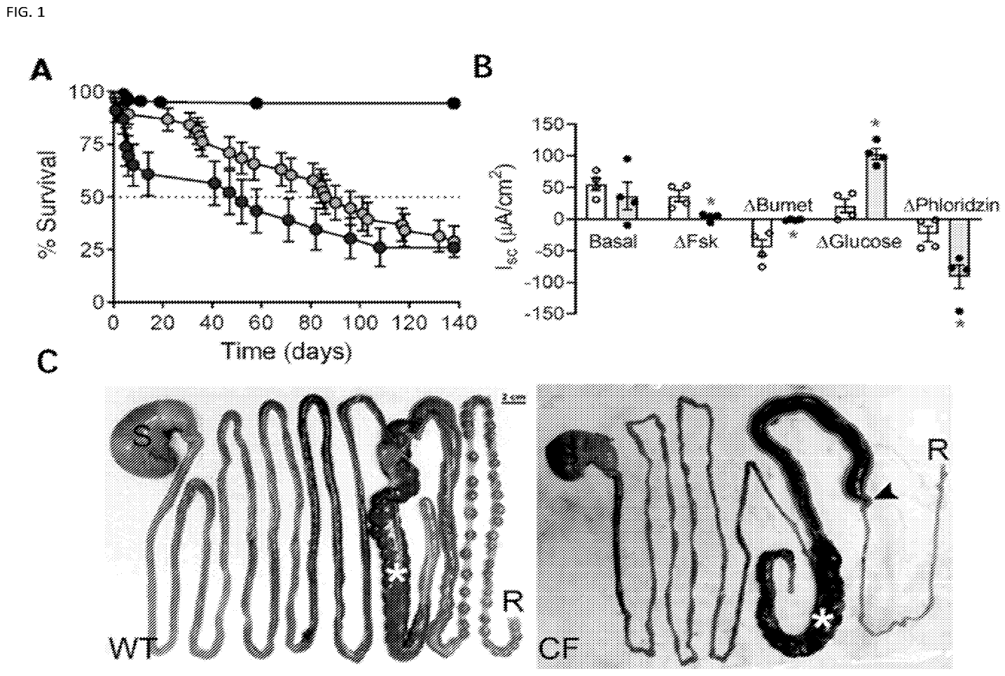

The CF rabbits we generated manifest most typical CF phenotypes as reported recently (13). Briefly, in comparison to non-CF littermates, CF rabbits grow slower and have lower body weight, and most CF rabbits die from intestinal obstruction, a condition that is improved with the use of laxative (FIG. 1). CF rabbits exhibited airway abnormalities in the bioelectric properties of the nasal and tracheal epithelia. Some animals, albeit a small percentage, developed spontaneous lung infections.

### CF Rabbits Develop Spontaneous Hepatobiliary Lesions and Abnormal Biliary Secretion.

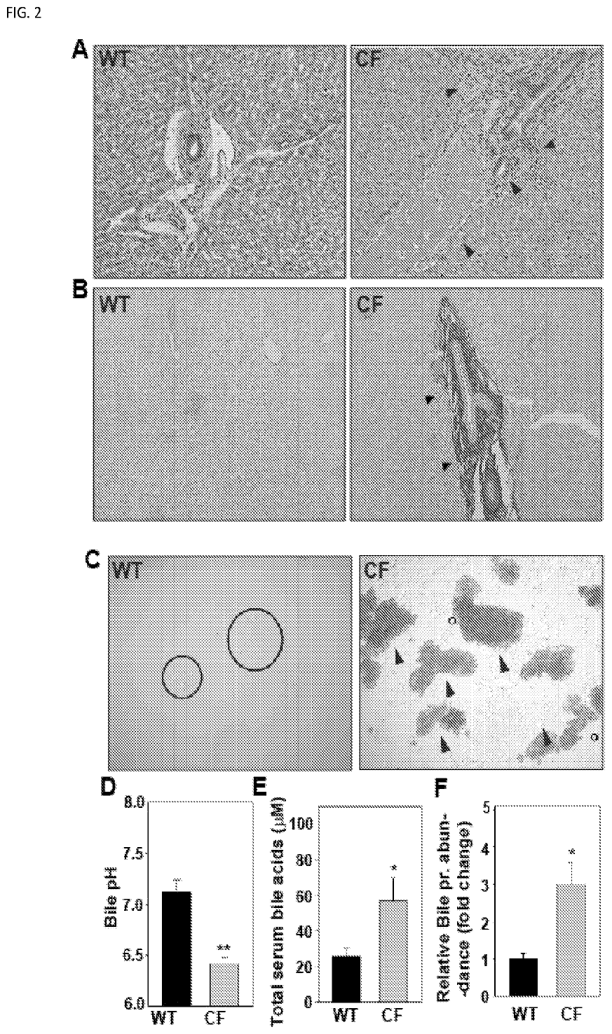

We examined the hepatic biliary system in APSE rabbits and identified typical CF-associated focal biliary fibrosis and cirrhosis around the bile duct accompanying with mucus plug, as shown by hematoxylin and eosin (H&E) staining and sinus-red staining of collagens (FIG. 2A-B). Further, CF rabbits showed abnormal biliary secretion. The bile collected from gallbladders of WT rabbits flowed easily, while the bile from CF rabbit gallbladders were thick and tenacious and exhibited crystal-like pigments (FIG. 2C). The bile pH of CF rabbits was significantly lower than that of WT (FIG. 2D). However, the levels of total serum bile acids of CF rabbits were much higher than those of WT (FIG. 2E). Additionally, the levels of serum bile proteins in CF rabbits were increased ˜3 times, compared to those of WT (FIG. 2F). These results revealed the major hepatic biliary phenotypes in CF rabbits that encapsulates human CFLD.

### CF Rabbits Display Micro-Gallbladder-Associated Pathology and Liver Injuries.

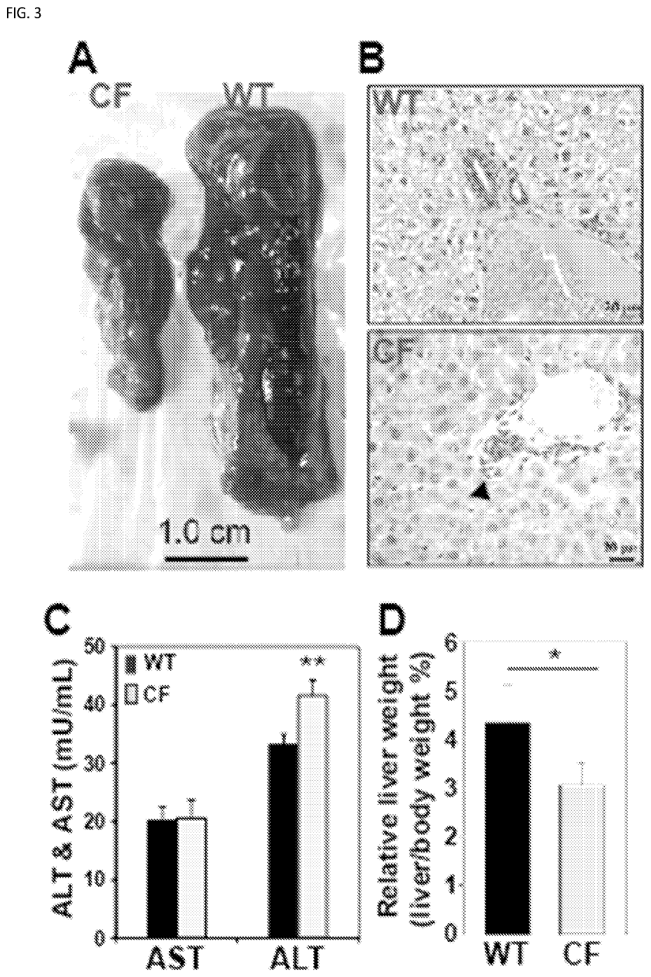

Among the hepatic manifestations observed in CF patients, gallbladder abnormality, namely micro-gallbladder, occurs frequently (15). The CF rabbits displayed micro-gallbladders (FIG. 3A) and cirrhosis in the biliary triad (FIG. 3B). The epithelial cells lining the bile triad ducts were disoriented and the ducts were stenosis in CF rabbits, but not in WT rabbits (FIG. 3B). These data indicated that CF rabbits have obstruction of intrahepatic bile ducts, causing focal biliary cirrhosis, inflammation, presumably due to overproduction of viscous mucus as shown in FIG. 2. Further, we examined serum levels of liver enzymes aspartate amino transferase (AST) and alanine aminotransferase (ALT). The levels of ALT and AST in CF rabbits were increased, compared to those in WT (FIG. 3C), indicating increased liver damage in CF rabbits. Consistently, relative liver weight (liver weight/body weight) of CF rabbits was reduced, compared to that of WT (FIG. 3D).

### CF Rabbits Exhibit NASH-Like Phenotype.

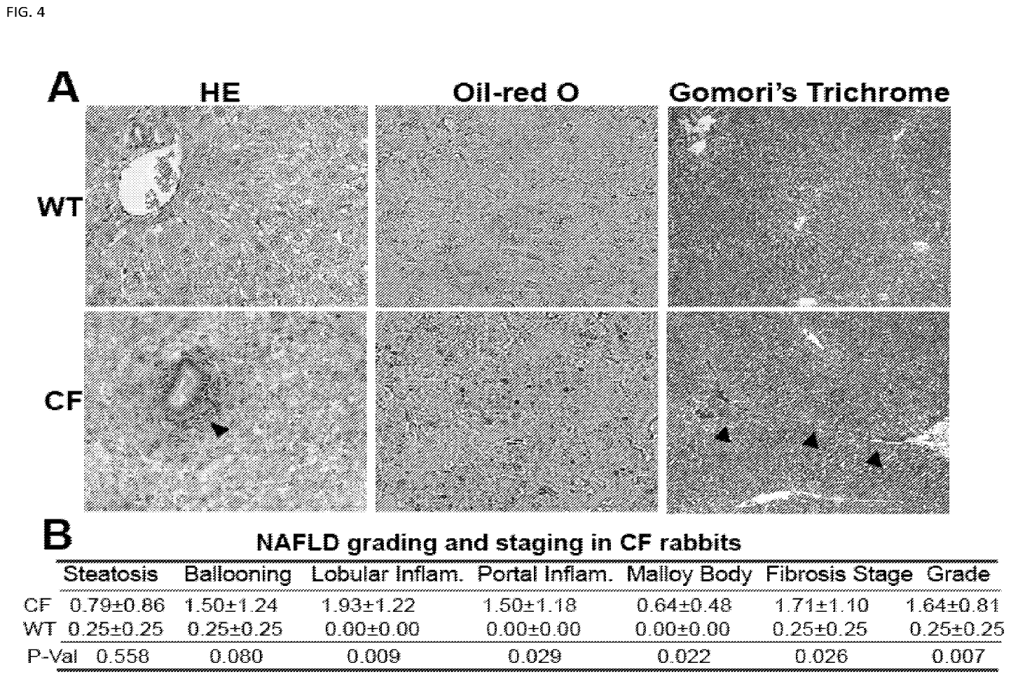

Many CF patients present with hepatic steatosis and non-alcoholic steatohepatitis (NASH) associated with multifactorial etiologies (15, 16). To assess NASH-associated activities in CF rabbits, we performed histological analyses with liver tissue sections from WT and CF rabbits of no more than 60 days. Based on H&E staining of the liver cellular structure, oil-red 0 staining of hepatic lipids, as well as Gomori's trichrome staining of the hepatic collagen deposition, we identified increased hepatic steatosis, lobular and portal inflammation, as well as perisinusoidal and portal fibrosis in the liver of CF rabbits, compared to WT control rabbits (FIG. 4A). Using the NAFLD grading and staging score system (17, 18) (FIG. 4B), we confirmed that approximately 36% CF rabbits of less than 60 days developed typical NASH phenotype, characterized by steatosis, hepatic inflammation, and perisinusoidal/portal fibrosis.

### CF Rabbits Show Elevated Lipid Profiles and Up-Regulation of Hepatic Metabolic Regulators.

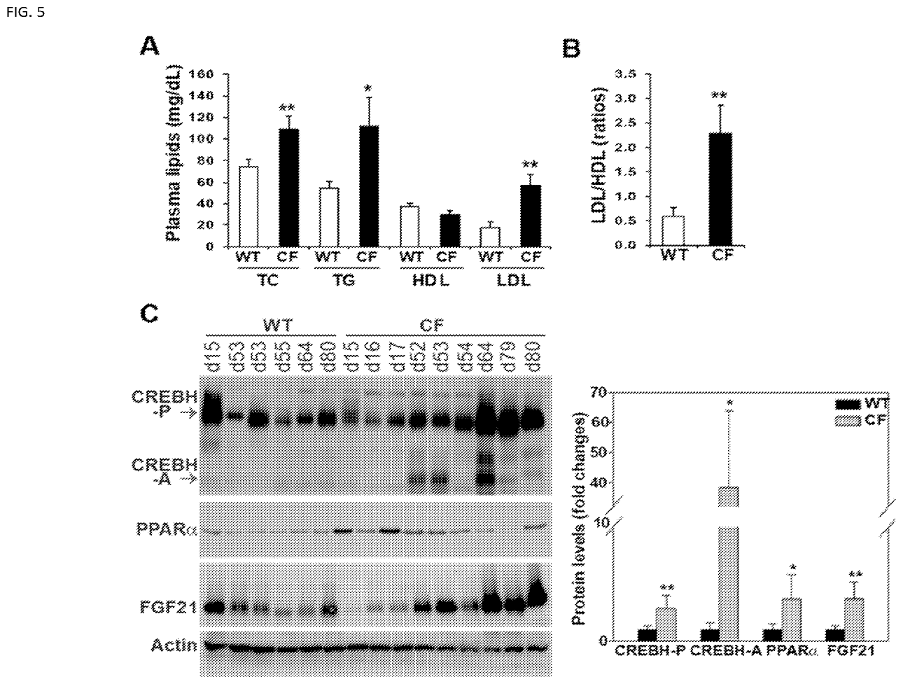

Adult CF patients develop metabolic risk factors typically associated with NASH, including diabetes mellitus or impaired glucose tolerance and hypertriglyceridemia, particularly with increasing age (15). Compared to WT control rabbits, CF rabbits exhibited significantly increased levels of plasma triglycerides (TG), total cholesterol (TC), and low-density lipoprotein cholesterol (LDL), but not high-density lipoprotein (HDL) (FIG. 5A-B), implicating a hyperlipidemia phenotype.

To gain mechanistic insights into the lipid phenotype of CF rabbits, we examined expression of several major metabolic regulators in the rabbit livers. Cyclic AMP-responsive element-binding protein H (CREBH) and peroxisome proliferator-activated receptor a (PPARα), two binary liver-enriched, stress-inducible transcriptional regulators, play important roles in regulating hepatic lipid and glucose metabolism (19-22). In comparison to WT control rabbits, expression levels of CREBH precursor (CREBH-P), activated CREBH protein (CREBH-A), and PPARα in livers of CF rabbits were increased with the advanced age (FIG. 5C). Fibroblast growth factor 21 (FGF21), whose expression is regulated by the CREBH-PPARα transcriptional complex, is a major hepatokine that drives mobilization of lipids and glucose in response to stress challenges (20). Similarly, expression levels of FGF21 in CF rabbit livers were increased in an age-dependent manner (FIG. 5C). Upregulation of the CREBH/PPARα/FGF21 regulatory axis may represent a feedback regulation of CF rabbit livers in an attempt to help the liver recover from the CFLD condition.

### CF Rabbits Show Glucose Intolerance and Diminished Glycogen Storage.

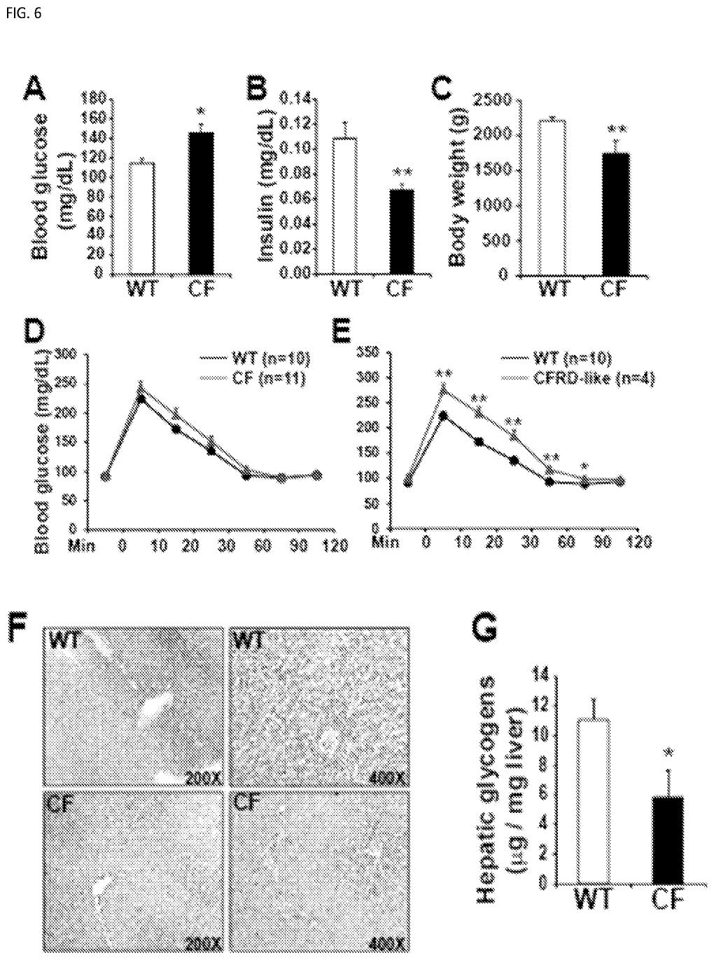

To assess the manifestation of CFLD in glucose homeostasis, we examined fasting blood glucose and insulin levels in WT and CF rabbits of no more than 60 days of age. The levels of blood glucose were increased, while blood insulin levels were decreased, in CF rabbits, compared to that of WT controls (FIG. 6A-B). Further, we performed an intravenous glucose tolerance test (IVGTT) with WT and CF rabbits. Upon administration of glucose, all the 11 CF rabbits examined had higher levels of blood glucose than the WT control rabbits (FIG. 6D). Among these animals, 4 CFLD-like rabbits, as determined by hepatic biliary lesion, micro-gallbladder, and NASH-like phenotype, showed significant glucose intolerance, as they displayed significantly reduced capability in glucose clearance, compared to the WT controls (FIG. 6E). Additionally, the body weights of CF rabbits were reduced, compared to those of WT rabbits (FIG. 6C). Next, we examined hepatic glycogen storage in WT and CF rabbits by both Periodic acid-Schiff (PAS) staining and quantitative enzymatic assays. Both approaches identified significantly reduced levels of hepatic glycogens in CF rabbit livers in comparison to WT rabbit livers (FIG. 6F-G). Of the 16 CF rabbits examined, 10 (62.5%) CF rabbits display depleted hepatic glycogen storage as indicated by PAS staining. Given the central role of hepatic glycogen storage in glucose homeostasis, hepatic glycogen depletion may account, at least partially, for the glucose intolerance observed in CF rabbits.

### ER Stress and Inflammatory Responses Prevail in CF Rabbit Livers.

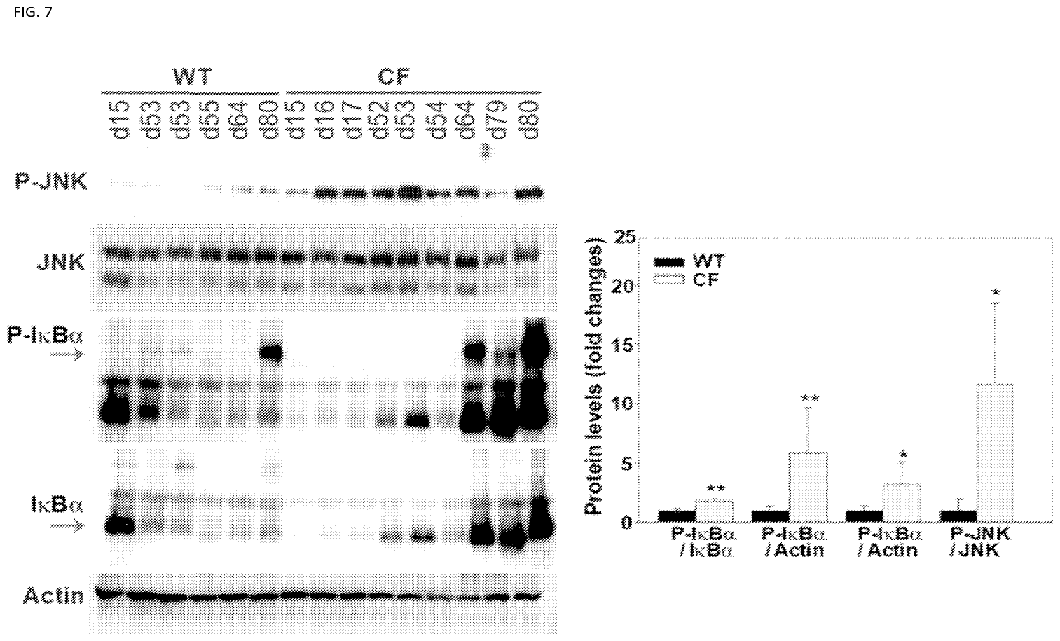

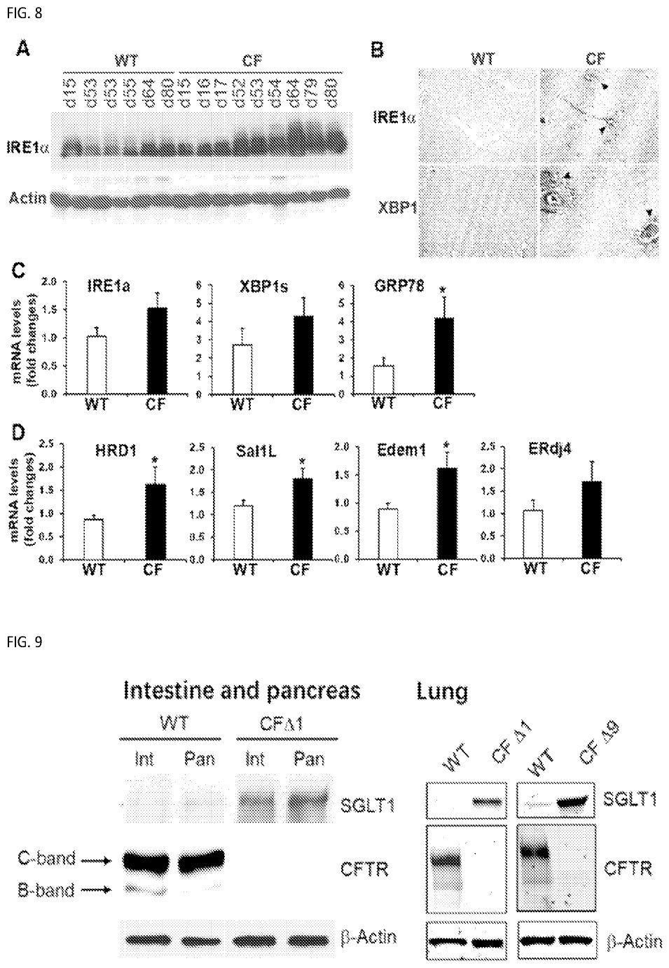

To understand the mechanistic basis underlying the CFLD phenotypes of CF rabbits, we examined activation of the major inflammatory pathways, mediated by JNK and NFκB, in CF rabbit livers. Compared to the WT, levels of phosphorylated JNK (P-JNK) and phosphorylated NFκB inhibitor (P-IκB), the indicators of JNK- and NFκB-mediated inflammatory pathways, were increased in rabbit livers in an age-dependent manner (FIG. 7). Next, we investigated activation of ER stress response or Unfolded Protein Response (UPR), the intracellular stress signaling that promotes inflammation and remodels metabolic homeostasis (23, 24), in the livers of WT and CF rabbits. Western blot analysis revealed age-dependent activation of ER stress response mediated by the primary ER stress sensor IRE1α and its downstream transcriptional activator XBP1 in CF rabbit livers (FIG. 8A). Immunohistochemistry (IHC) staining of rabbit liver tissue sections detected strong induction of IRE1α and XBP1 around the hepatic biliary ducts of CF, but not WT, rabbits of about 60 days of age (FIG. 8B). The activation of ER stress response through IRE1α and XBP1 was confirmed by quantitative real-time PCR (qPCR) analyses, which showed that mRNA expression levels of IRE1α, spliced Xbp1 (Xbp1s) and ER chaperone BiP/GRP78 (FIG. 8C) as well as the IRE1a/XBP1 targets in ER-associated degradation (ERAD) pathway including HRD1, SaliL, EDEM1, and ERdj4 (FIG. 8D). Together, these results revealed robust activation of ER stress and inflammatory responses in the livers of CF rabbits, particularly in those of more than 50 days of age.

### SGLT1 is Upregulated in CF Rabbit Tissues and Human CF Cells.

We first determined SGLT1 and SGLT2 expression levels in CF and WT rabbits. The SGLT2 expression, like that reported in humans, is largely restricted to the kidney, and there is no difference in between CF and WT rabbits; whereas SGLT1 expression is elevated in several CF relevant tissues including lung, pancreas and intestine (FIG. 9) and liver (FIG. 15A) in CF rabbits, suggesting that SGLT1 as a target for many CF affected organs including liver. We note that such upregulation is observed in both CFΔ1 (X mutation) and Δ9 (i.e. APSE) lines.

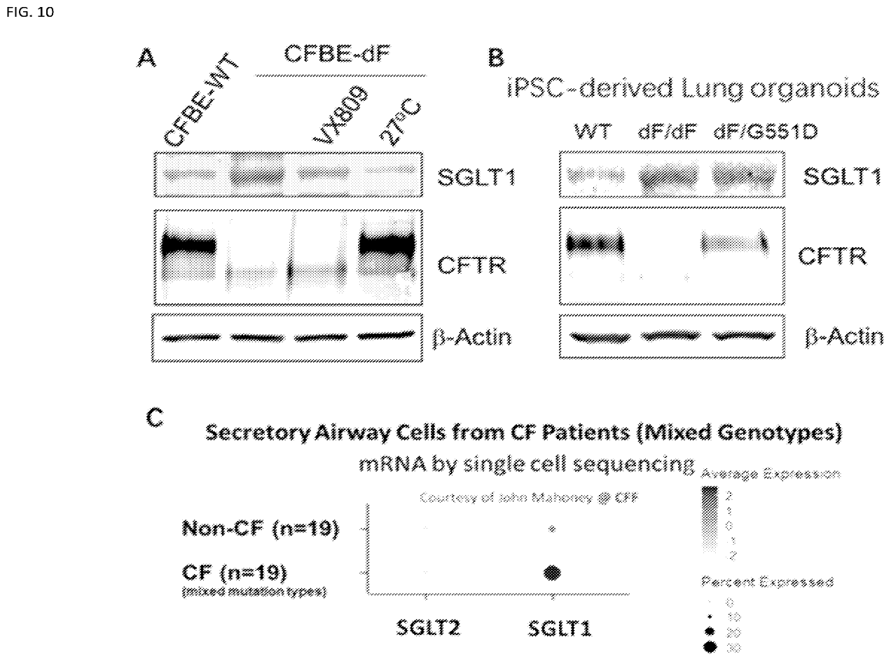

We also examined SGLT1 expression levels in human CF cells. The CFTR bandings in the CFBE cells are consistent with their genotypes. Similar to the findings in CF rabbits, the SGLT1 signals in CFBEs were reversely correlated with those of CFTR: high in the dF cells but low WT cells (FIG. 10A). Furthermore, pharmacological rescue of CFTR by VX809 or the low temperature rescue of CFTR at 27° C. in CFBE-dF cells were both associated with reduced SGLT1 levels (FIG. 10A). Consistently, in both dF/dF and dF/G551D lung organoids derived from patient iPSCs (FIG. 10B), the SGLT1 level was higher than that in the WT/WT. The pattern was also revealed in a recent project where single cell RNA sequencing of CF patient airway cells was conducted. SGLT1 was expressed not only at higher level, but also in higher percentage of cells in the secretory airway cells from CF subjects (mixed genotypes) than those from non-CF; whereas SGLT2 is only expressed at trace amount (FIG. 10C).

### Sota Improves Glucose Tolerance in CF Rabbits.

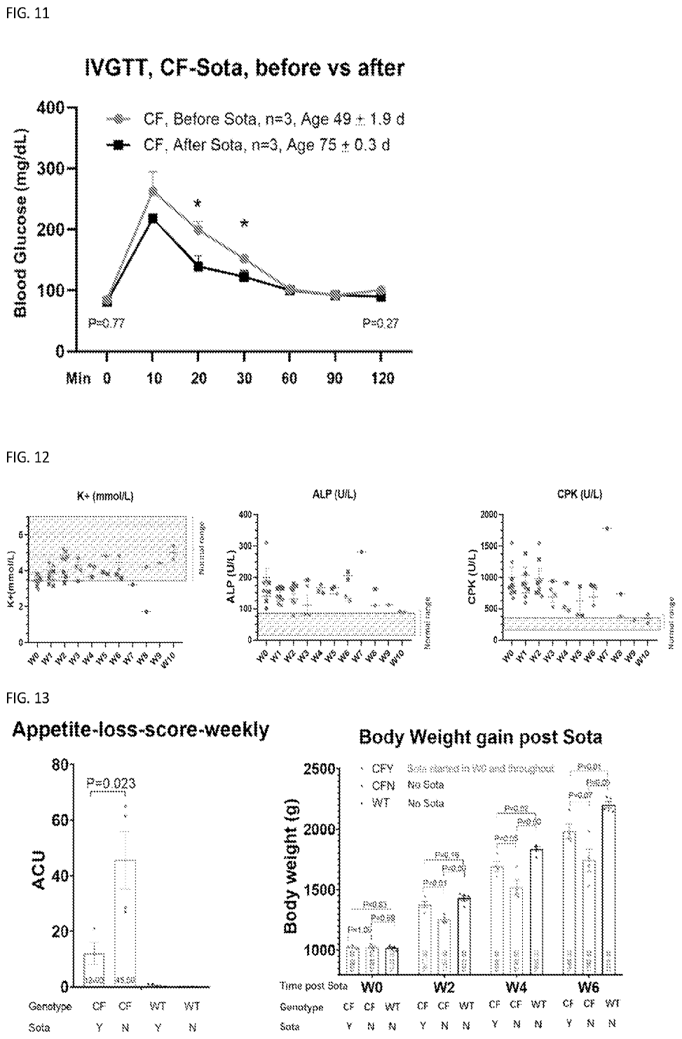

We proceeded to treat APSE rabbits with Sota (15 mg/kg/day) by daily gavage for 4 weeks. As expected, urine glucose level spiked upon Sota treatment and returned to normal when the drug was retrieved. The IVGTT tests were performed 2 weeks after drug treatment. Sota-treated animals showed a higher blood glucose elimination rate than those in the non-treatment group (FIG. 11), demonstrating Sota's beneficial effects on glucose metabolism in CF rabbits.

### Sota Improves Liver Function Parameters in CF Rabbits.

We examined blood chemistry in APSE CF rabbits, and found that CF rabbits presented many abnormalities in metabolic parameters, as well as serum ALP, CPK, among others. To evaluate if Sota has any effects on the blood chemistry parameters of CF rabbits, we treated five CFΔ9 rabbits with Sota for 10 weeks. CFΔ9 rabbits in the control group (n=5) did not receive any Sota treatment. Surprisingly, the Sota treatment significantly improved the imbalanced/abnormal parameters such as K+, ALP and CPK (FIG. 12), as well as Triglycerides (TG), Glucose, and total Cholesterol. In the control group animals, these parameters gradually worsened, whereas in the Sota group, these parameters returned to the normal ranges (grey boxes, FIG. 12). These findings implicate that Sota treatment benefits CF rabbits beyond the glucose metabolism, such as on electrolyte imbalance, lipid metabolism and liver function.

### Sota Treatment Elongates CF Rabbit Lifespan.

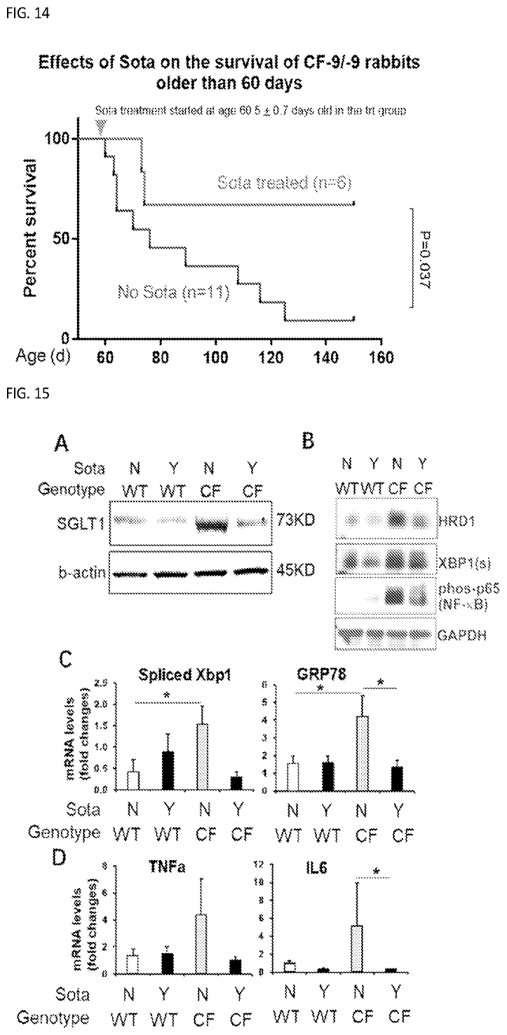

The most unexpected and significant finding is that Sota treatment elongated lifespan of APSE CF rabbits. With Sota, the APSE rabbits treated with Sota (n=6) lived significantly longer than those without (n=11) (FIG. 14). This result indicates the SGLT inhibitor drugs bring systemic benefits to CF individuals.

### Sota Alleviates ER Stress Response and Inflammation Markers in CF Rabbit Livers.

The improved live function parameters in APSE CF rabbits treated Sota prompted us to exam SGLT1 in the liver. Consistent with findings in other tissues, SGLT1 is upregulated in CF rabbit livers, but not in those treated with Sota (FIG. 15A). Notably, Sota treatment reduced levels of the major ER stress marker spliced Xbp1 mRNA (XBP1s) and the major inflammatory response marker phosphorylated NFκB p65 protein (FIG. 15B). qPCR analysis confirmed that the levels of the mRNAs encoding XBP1s and the master UPR regulator BiP/GRP78 (FIG. 15C) as well as the pro-inflammatory cytokines TNFα and IL6 were reduced in CF rabbits treated with Sota, compared to those of the CF rabbits without Sota treatment (FIG. 15D). Intriguingly, we also found that Sota treatment reduced levels of ERAD-associated E3 ligase HRD1, the target of IRE1a/XBP1 UPR pathway (25) (FIG. 15B), suggesting a potential role of ERAD in CFLD.

### SGLT1 is Highly Expressed in the Hepatocytes Under Disease Conditions.

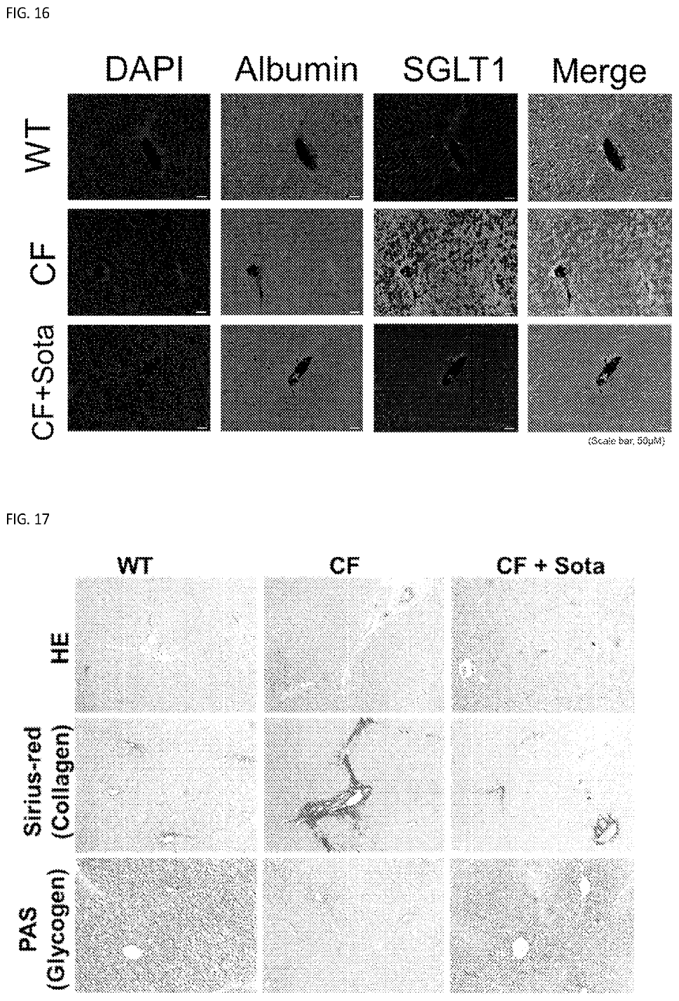

SGLT1 is known to express in epithelial cells but not in hepatocytes. Unexpectedly, experimetns were conducted that revealed that while SGLT1 is not expressed in WT rabbit hepatocytes, it is highly expressed in CF rabbit hepatocytes (FIG. 16). As shown in FIG. 16, SGLT1 signals colocalized with the signals of hepatocyte marker Albumin.

### Sota Alleviates NASH-Like Phenotype in CF Rabbit Livers.

Experiments were conducted that investigated if Sota treatment has any effects on the NASH-like phenotype in CF rabbit livers. Histological analysis demonstrated that livers of Sota treated CF rabbits, as compared to CF rabbits without Sota, displayed much less extent of fibrosis, revealed by the Sirius-red staining of the collage (FIG. 17 central row), but enhanced glycogen storage capacity, reveled by the PAS staining (FIG. 17 bottom row).

NASH relevant parameters including lobular inflammation, portal inflammation, lobular necroinflammation, Mallory bodies and fibrosis stage, determined by a certified pathologist, were all significantly improved in Sota treated CF rabbit livers (Table 1). These data indicate that Sota alleviates NASH-like phenotype in CF rabbit livers.

TABLE 1
NASH scores (Mean ± SEM) of WT and CF rabbits with or w/o Sota.

| | | Portal | Lobular | | Fibrosis stage |
|---|---|---|---|---|---|
| | Lobular inflammation | inflammation | necroinflammation | Mallory bodies | (0-4) |
| WT | 0.165 ± 0.09 | 0.08 ± 0.08 | 0.00 ± 0.00 | 0.00 ± 0.00 | 0.33 ± 0.13 |
| CF | 2.835 ± 0.09 | 1.00 ± 0.00 | 1.33 ± 0.27 | 0.75 ± 0.16 | 3.08 ± 0.24 |
| CF-Sota | 0.583 ± 0.15 | 0.41 ± 0.16 | 0.16 ± 0.16 | 0.08 ± 0.08 | 1.00 ± 0.19 |
| P (CF vs WT) | 1.07E−06 | 3.15E−05 | 2.68E−03 | 3.38E−03 | 6.98E−05 |
| P (CF vs CF-Sota) | 1.96E−05 | 0.01 | 0.01 | 0.01 | 5.61E−04 |

### Sota Normalizes Bile Acid (BA) Profiles in Liver of CF Rabbits.

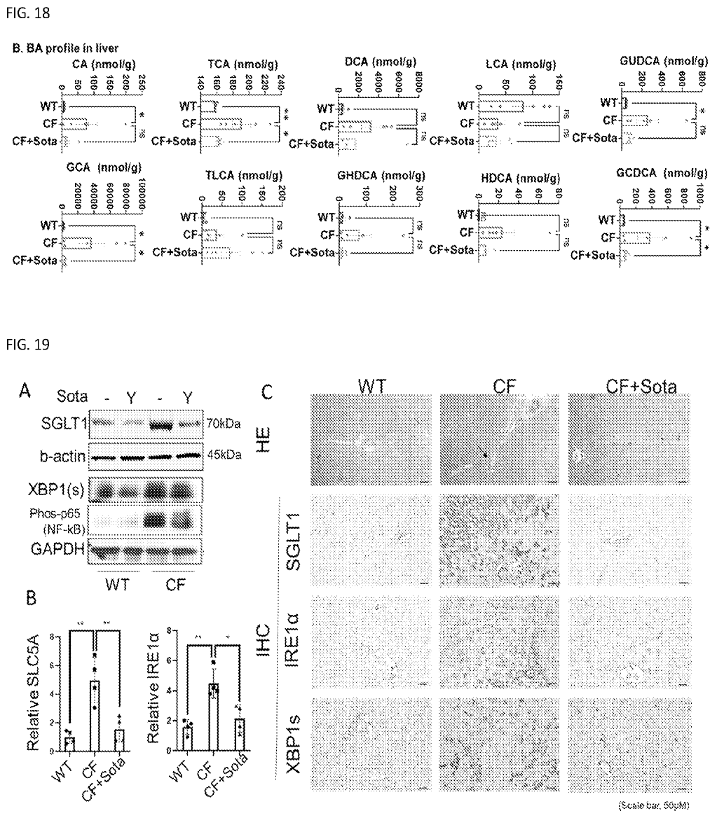

It is known that bile acid dysregulation contributes to CFLD. Experiments were conducted that collected samples from WT and CF rabbits (with or w/o Sota) and analyzed at the University of Michigan Metabolomics Core. The bile acid-targeted metabolomics analysis showed that over 80% of BA species, including both primary and secondary ones were altered in the liver samples (FIG. 18) of the CF rabbits. Importantly, Sota treatment to the CF rabbits normalized BA profiles in the liver (FIG. 18).

### Sota Alleviates ER Stress Response in CF Rabbit Livers and Other Organs.

In the liver, SGLT1 is upregulated in those of CF rabbits, but not in those treated with Sota (FIG. 19A). Notably, Sota treatment reduced levels of the ER stress marker XBP1s, as well as inflammation marker phosphorylated NFκB p65 protein (FIG. 19A). qPCR and IHC analysis confirmed these findings (FIGS. 19B & C). These data indicate potential roles of ER stress and inflammation in CFLD pathogenesis, and the mechanism of action for the beneficial effects of Sota's on CFLD is by suppressing ER stress response and hepatic inflammation.

### XBP1s is a Transcription Factor of SLC5A1.

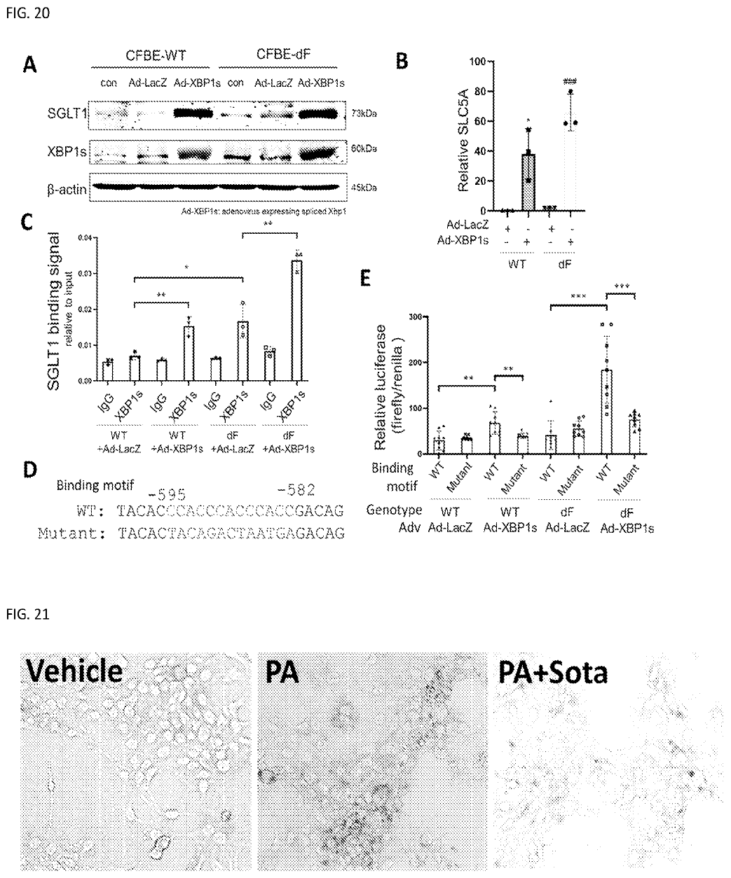

It was hypothesized that the ER stress trans-activator XBP1s is a transcription factor for the SLC5A1 gene that encode SGLT1. To test this, experiments were conducted using bronchial epithelial cells carrying the dF (CFBE-dF) or the WT CFTR (CFBE-WT). It was shown that overexpression of)(BP's led to upregulation of SLC5A1 at the transcription level and SGLT1 at the protein level in both WT and dF cells (FIGS. 20A&B). Chromatin immunoprecipitation (ChIP) assay demonstrated that XBP1s binds to the SLC5A1 gene promoter (FIG. 20C). We further determined that the putative binding motif is at −590 bp position (FIG. 20D). Mutation of this motif abolished the XBP1s binding as shown in the luciferase assay (FIG. 20E).

### Sota Attenuates ER Stress Induced Steatosis in an In Vitro NASH Model.

Given that ER stress transducer XBP1s is a transcription factor of SGLT1, and the observation that Sota inhibition attenuates ER stress in CF rabbit liver, it was hypothesized that under pathological conditions, including but not limited to CF, SGLT1 is activated in hepatocytes, leading to the glucose hyperabsorption, elevated intracellular glucose levels, and subsequent activated ER stress and inflammatory responses. As such, SGLT1 inhibition may provide benefits to a variety of liver diseases through the attenuating ER stress mechanism.

To test this, experiments were conducted that established a palmitate (PA) induced NASH model using human hepatocyte cell line Huh7. It is known that PA, a saturated fatty acid, will induce haptic steatosis associated with ER stress (26). The Huh7 cells were treated with PA at 10 ug/ml for 36 h, with or without Sota (5 μg/ml) supplementation in the culture medium. Oil-red staining was employed to evaluate the extent of steatosis. It was shown that Sota treatment significantly reduced the extent of steatosis, as indicated by the Sirius-red staining (FIG. 21).

Hypothesized Mechanism of Action by which SGLT1 Inhibition Benefits Inflammatory Liver Diseses.

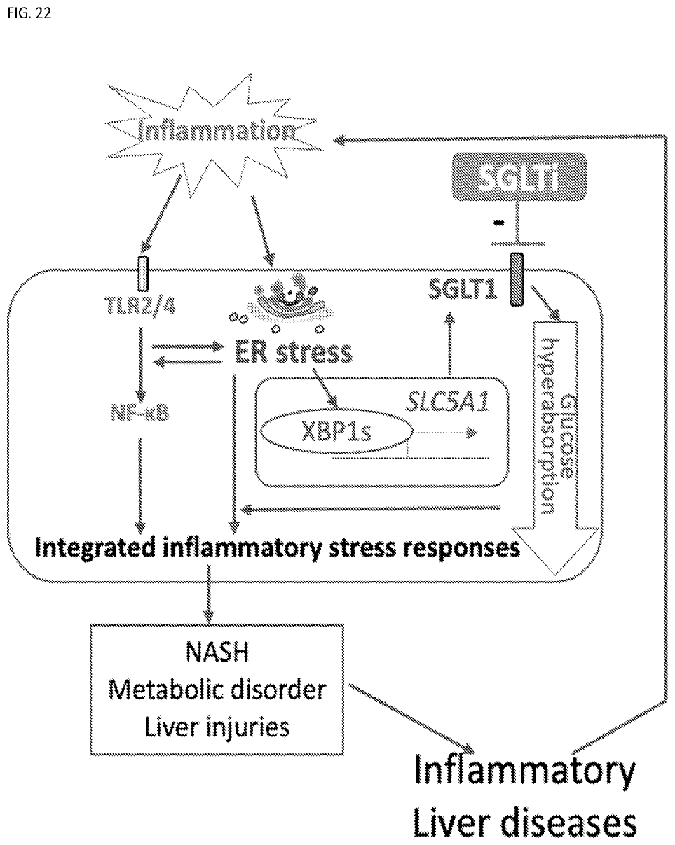

ER stress is a major contributor to many liver diseases. It was hypothesized that ER stress/inflammation→SGLT1 upregulation→aggravated ER stress/inflammation forms a vicious cycle in liver diseases in general, and that pharmacological disruption of this cycle represents a therapeutic strategy to treat liver diseases (FIG. 22).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. The following references denoted throughout the application by numerical reference are herein incorporated by references throughout their entireties:
1. D. A. Stoltz, D. K. Meyerholz, M. J. Welsh, Origins of cystic fibrosis lung disease. N Engl J Med 372, 351-362 (2015).
2. R. D. Baker, S. S. Baker, Cystic Fibrosis-related Liver Disease: The Next Challenge. J Pediatr Gastroenterol Nutr 71, 421-422 (2020).
3. F. S. Collins, Realizing the Dream of Molecularly Targeted Therapies for Cystic Fibrosis. N Engl J Med 381, 1863-1865 (2019).
4. K. Staufer, Current Treatment Options for Cystic Fibrosis-Related Liver Disease. Int J Mol Sci 21 (2020).
5. R. Fiorotto et al., Animal models for cystic fibrosis liver disease (CFLD). Biochim Biophys Acta Mol Basis Dis 1865, 965-969 (2019).
6. R. Sano, Y. Shinozaki, T. Ohta, Sodium-glucose cotransporters: Functional properties and pharmaceutical potential. J Diabetes Investig 10.1111/jdi.13255 (2020).
7. G. Gyimesi, J. Pujol-Gimenez, Y. Kanai, M. A. Hediger, Sodium-coupled glucose transport, the SLC5 family, and therapeutically relevant inhibitors: from molecular discovery to clinical application. Pflugers Arch 472, 1177-1206 (2020).
8. E. D. Deeks, Sotagliflozin: A Review in Type 1 Diabetes. Drugs 79, 1977-1987 (2019).
9. M. F. Hassanabad, Z. F. H. Abad, Are SGLT2 inhibitors joining the mainstream therapy for diabetes type 2? Diabetes Metab Syndr 13, 1893-1896 (2019).
10. R. J. Chilton, Effects of sodium-glucose cotransporter-2 inhibitors on the cardiovascular and renal complications of type 2 diabetes. Diabetes Obes Metab 22, 16-29 (2020).
11. D. L. Bhatt et al., Sotagliflozin in Patients with Diabetes and Recent Worsening Heart Failure. N Engl J Med 384, 117-128 (2021).
12. D. L. Bhatt et al., Sotagliflozin in Patients with Diabetes and Chronic Kidney Disease. N Engl J Med 384, 129-139 (2021).
13. J. Xu et al., Phenotypes of CF rabbits generated by CRISPR/Cas9-mediated disruption of the CFTR gene. JCI Insight 6 (2021).
14. D. Yang et al., Production of CFTR-ΔF508 Rabbits. Front Genet 11 (2021).
15. S. Sakiani, D. E. Kleiner, T. Heller, C. Koh, Hepatic Manifestations of Cystic Fibrosis. Clin Liver Dis 23, 263-277 (2019).
16. R. Fiorotto, M. Strazzabosco, Pathophysiology of Cystic Fibrosis Liver Disease: A Channelopathy Leading to Alterations in Innate Immunity and in Microbiota. Cell Mol Gastroenterol Hepatol 8, 197-207 (2019).
17. E. M. Brunt, Nonalcoholic steatohepatitis: definition and pathology. Semin Liver Dis 21, 3-16 (2001).
18. Z. Zheng et al., Exposure to fine airborne particulate matters induces hepatic fibrosis in murine models. J Hepatol 63, 1397-1404 (2015).
19. C. Zhang et al., Endoplasmic reticulum-tethered transcription factor cAMP responsive element-binding protein, hepatocyte specific, regulates hepatic lipogenesis, fatty acid oxidation, and lipolysis upon metabolic stress in mice. Hepatology 55, 1070-1082 (2012).
20. H. Kim et al., Liver-enriched transcription factor CREBH interacts with peroxisome proliferator-activated receptor alpha to regulate metabolic hormone FGF21. Endocrinology 155, 769-782 (2014).
21. H. Kim, Z. Zheng, P. D. Walker, G. Kapatos, K. Zhang, CREBH Maintains Circadian Glucose Homeostasis by Regulating Hepatic Glycogenolysis and Gluconeogenesis. Mol Cell Biol 37 (2017).
22. S. Kersten, R. Stienstra, The role and regulation of the peroxisome proliferator activated receptor alpha in human liver. Biochimie 136, 75-84 (2017).
23. C. Hetz, K. Zhang, R. J. Kaufman, Mechanisms, regulation and functions of the unfolded protein response. Nat Rev Mol Cell Biol 21, 421-438 (2020).
24. K. Zhang, R. J. Kaufman, From endoplasmic-reticulum stress to the inflammatory response. Nature 454, 455-462 (2008).
25. K. Zhang et al., The unfolded protein response transducer IRE1alpha prevents ER stress-induced hepatic steatosis. EMBO J 30, 1357-1375 (2011).
26. C. S. Achard, D. R. Laybutt, Lipid-induced endoplasmic reticulum stress in liver cells results in two distinct outcomes: adaptation with enhanced insulin signaling or insulin resistance. Endocrinology 153, 2164-2177 (2012).

What is claimed is:

1. A composition comprising a pharmaceutical agent capable of inhibiting SGLT-1 activity.

2. The composition of claim 1, wherein the pharmaceutical agent is a small molecule, an antibody, nucleic acid molecule (e.g., siRNA, antisense oligonucleotide, an aptamer), or a mimetic peptide.

3. The composition of claim 1, wherein the pharmaceutical agent is selected from Phlorizin, Canagliflozin ((2S,3R,4R,5S,6R)-2-{3-[5-[4-Fluoro-phenyl)-thiophen-2-ylmethyl]-4-Xmethyl- -phenyl}-6-hydroxymethyl-tetrahydro-pyran-3,4,5-triol), Dapagliflozin ((2S,3R,4R,5S,6R)-2-[4-chloro-3-(4-ethoxybenzyl)phenyl]-6-(hydroxymethyl)-tetrahydro-2H-pyran-3,4,5-triol), Empagliflozin ((2S,3R,4R,5S,6R)-2-[4-chloro-3-[[4-[(3S)-oxolan-3-yl]oxyphenyl]methyl]phenyl]-6-(hydroxymethyl)oxane-3,4,5-triol), Remogliflozin (5-methyl-4-[4-(1-methylethoxy)benzyl]-1-(1-methylethyl)-1H-pyrazol-3-yl 6-O-(ethoxycarbonyl)-(3-D-glucopyranoside), Sergliflozin (2-(4-methoxybenzyl)phenyl 6-O-(ethoxycarbonyl)-(3-D-glucopyranoside), and Tofogliflozin ((1S,3′R,4′S,5′S,6′R)-6-(4-Ethylbenzyl)-6′-(hydroxymethyl)-3′,4′,5′,6′-te-trahydro-3H-spiro[2-benzofuran-1,2′-pyran]-3′,4′,5′-triol hydrate (1:1)), and Sotagliflozin (2S,3R,4R,5S,6R)-2-(4-chloro-3-(4-ethoxybenzyl)phenyl)-6-(methylthio)tetrahydro-2H-pyran-3,4,5-triol (LX4211), or a pharmaceutically acceptable salt thereof.

4. A method for inhibiting the activity of SGLT-1 in a subject, comprising administering to the subject a composition as recited in claim 1.

5. The method of claim 4, wherein the subject is suffering from or at risk of suffering from one or more liver diseases and/or conditions characterized by one or more of
increased SGLT-1 activity;
increased endoplasmic reticulum (ER) stress response; and
increased hepatic inflammation.

6. The method of claim 5, wherein the one or more liver diseases and/or conditions characterized by one or more of increased SGLT-1 activity, increased ER stress, and/or increased hepatic inflammation are selected from CFLD, spontaneous hepatobiliary lesions, increased liver damage, NASH activities, altered lipid and glucose homeostasis, alpha-1 antitrypsin deficiency (AATD), chronic viral hepatitis, cholestatic liver diseases, alcohol-induced liver injury or alcoholic fatty liver disease, hyperhomocysteinemia, liver ischemia/reperfusion injury, drug-induced liver injuries, and hepatocellular carcinoma (HCC).

7. The method of claim 4, wherein the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

8. A method for treating, ameliorating and/or preventing in a subject one or more symptoms related to liver diseases and/or conditions associated with one or more of
increased SGLT-1 activity,
increased endoplasmic reticulum (ER) stress response, and
increased hepatic inflammation;
comprising administering to the subject a composition as recited in claim 1.

9. The method of claim 8, wherein the subject is suffering from or at risk of suffering from or at risk of suffering from one or more liver diseases and/or conditions characterized by increased SGLT-1 activity, increased ER stress, and/or increased hepatic inflammation (e.g., CFLD, spontaneous hepatobiliary lesions, increased liver damage, NASH activities, altered lipid and glucose homeostasis, alpha-1 antitrypsin deficiency (AATD), chronic viral hepatitis, cholestatic liver diseases, alcohol-induced liver injury or alcoholic fatty liver disease, hyperhomocysteinemia, liver ischemia/reperfusion injury, drug-induced liver injuries, and HCC).

10. The method of claim 8, the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

11. The method of claim 8, wherein the one or more symptoms are selected from diminished liver function, hepatic and metabolic dysregulation, and hepatic ER stress and related inflammatory responses.

12. A method for treating, ameliorating and/or preventing CFLD in a subject, comprising administering to the subject a composition as recited in claim 1.

13. The method of claim 12, wherein the subject is suffering from or at risk of suffering from or at risk of suffering from CFLD.

14. The method of claim 12, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

15. A method for treating, ameliorating and/or preventing spontaneous hepatobiliary lesions in a subject, comprising administering to the subject a composition as recited in claim 1.

16. The method of claim 15, wherein the subject is suffering from or at risk of suffering from or at risk of suffering from spontaneous hepatobiliary lesions.

17. The method of claim 15, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

18. A method for treating, ameliorating and/or preventing liver damage in a subject, comprising administering to the subject a composition as recited in claim 1.

19. The method of claim 18, wherein the subject is suffering from or at risk of suffering from or at risk of suffering from liver damage.

20. The method of claim 18, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

21. A method for treating, ameliorating and/or preventing NASH in a subject, comprising administering to the subject a composition as recited in claim 1.

22. The method of claim 21, wherein the subject is suffering from or at risk of suffering from or at risk of suffering from NASH.

23. The method of claim 22, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

24. A method for treating, ameliorating and/or preventing altered lipid and glucose homeostasis in a subject, comprising administering to the subject a composition as recited in claim 1.

25. The method of claim 24, wherein the subject is suffering from or at risk of suffering from or at risk of suffering from altered lipid and glucose homeostasis.

26. The method of claim 24, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

27. A method for treating, ameliorating and/or preventing hepatic ER stress and related inflammatory responses in a subject, comprising administering to the subject a composition as recited in claim 1.

28. The method of claim 27, wherein the subject is suffering from or at risk of suffering from or at risk of suffering from hepatic ER stress and related inflammatory responses.

29. The method of claim 27, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

30. A method for treating, ameliorating and/or preventing hepatic and metabolic dysregulation in a subject, comprising administering to the subject a composition as recited in claim 1.

31. The method of claim 30, wherein the subject is suffering from or at risk of suffering from or at risk of suffering from hepatic and metabolic dysregulation.

32. The method of claim 30, wherein the the administration of the pharmaceutical agent results in one or more of attenuation of hepatic and metabolic dysregulation, mitigation of hepatic ER stress and inflammatory responses, improvement of liver function, restoration of blood glucose homeostasis, increasing of body weight, and increasing of life span.

33. A kit comprising (1) a composition as recited in claim 1, (2) a container, pack, or dispenser, and (3) instructions for administration.

34. A pharmaceutical composition comprising a composition as recited in claim 1.
